(19) United States
(12) Patent Application Publication
(10) Pub. No.: US 2007/0065386 A1
(43) Pub. Date: Mar. 22, 2007

(54) Use of oligosaccharides in cosmetic, food or dermatological compositions for stimulating the differentiation of epidermal cell

(76) Inventors: Christian Lubrano, Issy-Les-Moulineaux (FR); Nathalie Le Batteux, Issy-Les-Moulineaux (FR)

Correspondence Address: BACHMAN & LAPOINTE, P.C., 900 CHAPEL STREET, SUITE 1201, NEW HAVEN, CT, 06510, US

(21) Appl. No.: 10/555,532

(22) PCT Filed: May 6, 2004
(86) PCT No.: PCT/FR04/01101

§ 371 Date: Oct. 6, 2006

(30) Foreign Application Priority Data
May 6, 2003 (FR) 03/05511

(51) Int. Cl. A61K 8/73; A61K 31/715

(52) U.S. Cl. 424/70.13; 514/54; 514/61

(57) ABSTRACT

The present invention related to a method for preventing or treating skin disorders comprising administering to a subject a dermatological composition comprising oligogalacturonides.

This application is a continuation of PCT Application No. PCt/FR2004/001101, filed May 6, 2004, which in turn claims the benefit of French Application 03/05511 filed May 6, 2003, all of which are incorporated herein by reference.

### BACKGROUND

(1) Field of the Invention

The present invention relates to the fields of cosmetics and foodstuffs, and particularly to a method for preventing or treating skin disorders by administrating a composition comprising oligogalacturonides as an agent for stimulating cell differentiation, mainly epidermal cells differentiation.

(2) Prior Art

Pectin-derived oligogalacturonides are described in the abstract of Japanese patent Vol. 1997 no. 6 in the name of POLA CHEM IND Inc. as a free-radical trapping agent that intends to trap the activated oxygen generated in the body.

### SUMMARY OF THE INVENTION

The invention relates to a method for preventing or treating skin disorders comprising administrating to a subject a dermatological composition comprising oligogalacturonides.

In various embodiment, said methods are for treating or preventing epidermal disorders, and said dermatological composition stimulates the differentiation of epidermal cells, preferably the differentiation of keratinocytes.

In other various embodiments, said methods comprise administrating a dermatological composition comprising, in their dry equivalent, oligogalacturonides in a quantity between around 0.001% and around 1%, preferably around 0.01% in weight in relation to the total weight of the composition.

In others various embodiments, said methods comprise administrating a dermatological composition comprising oligogalacturonides having a degree of polymerization comprised between around 1 and around 5.

In another various embodiment, said dermatological composition comprises oligogalacturonides obtained by hydrolysis of a pectin, the said hydrolysis being performed by an enzymatic cocktail containing pectinases having mainly polygalacturonase, methyl esterase and polygalacturonase lyase activities.

In other various embodiments, these methods can be used in the fields of cosmetics and foodstuffs as means for improving the appearance and feel of the skin by fighting against skin irregularities and/or improving the radiance of the complexion and/or smoothing the skin of the face and/or or of the body, and/or improving the quality of the skin tissue, and/or stimulating the cell renewal process, mainly epidermal.

### DETAILED DESCRIPTION OF THE PREFERRED EMBODIMENT(S)

The Applicant has now highlighted a new property of oligogalacturonides, and shows how they act as an agent that stimulates differentiation of skin cells, mainly epidermal and particularly keratinocytes. Their use in the fields of cosmetics and foodstuffs and for the preparation of dermatological compositions makes it possible to prevent or treat skin disorders, mainly epidermal. Oligogalacturonides are particularly useful in the fields of cosmetics, foodstuffs and for the preparation of dermatological compositions that are designed to fight against skin irregularities and/or to improve the radiance of the complexion and/or to smooth the skin of the face and/or of the body, and/or to improve the quality of the skin tissue and/or to stimulate the cell renewal process, mainly epidermal and particularly keratinocytes. Oligogalacturonides are also useful for fighting against skin aging, particularly epidermal micro aging, and have an action for preventing or reducing wrinkles and fine wrinkles. They have a gentle exfoliating action.

The oligogalacturonides added to the compositions according to the invention are preferably obtained by enzymatic hydrolysis of pectin.

Pectin consists of a principal chain called pectic acid comprising a sequence of galacturonic acid type sugars. The constitutive sugars can be methylated or esterified, and the proportion of transformed sugars is typical of a vegetable species. The principal chain is sometimes interrupted by the insertion of a side chain of neutral sugars, such as rhamnose or glucose.

Oligogalacturonides, formerly known as oligosaccharides, have been described as veritable vegetable hormones (Darvill et al., Glycobiology, vol 2, no. 3, pp 181-198, 1992). They can be prepared by hydrolysis of the pectin, with the size or degree of polymerisation of the oligogalacturonides depending on the conditions of the hydrolysis reaction.

The Applicant has now perfected a method for preparing oligogalacturonides that provides an industrial product, which is effective as a cosmetic or dermatological agent.

Numerous pectins are available commercially in large quantity and variable quality. These are often standardized pectins that frequently contain added sugars to enable homogenisation of the viscosities among the batches. In fact, the first use of these pectins is linked to their gelling properties and as viscosity agents.

The oligogalacturonides employed in the compositions of the invention are preferably prepared by hydrolysis of pectin having a low degree of methylation and esterification to be as close as possible to polygalacturonic acid

The pectins of the type HERBSTREITH™ FOX Classic AU 910™ obtained from apples (pectin type HB AU) are an example of such a pectin.

The enzymes used for the hydrolysis of pectin are of the industrial type frequently used in the fruit juice processing industry. These are cocktails of enzymes conceived to cut the membranal pectins and thereby enable better extraction of the fruit juices by making the structures fragile prior to pressing. They also make possible clarification of the juices in which turbidity, often linked to pectins, is not desirable. The enzyme marketed by the company LYVEN as Clarification granulated is an example of such an enzyme. The enzyme cocktail comprises pectinases having principally polygalacturonase, methyl esterase and polygalacturonase lyase activities.

A preferred method for preparing the oligogalacturonides according to the invention comprises the following steps:
hydrolysis of a pectin solution at a concentration of 0.1% to 10%, preferably 1%, with a pH of around 4.5 by adding to the said pectin solution an enzyme solution comprising 10 to 1000 and preferably 100 units of polygalacturonase so as to obtain between 1 and 10 and preferably 4 units of pectinase in the final solution;
stopping the hydrolysis;
separating the polymers with a high molecular weight and recovering the oligogalacturonides.

Hydrolysis is advantageously performed at 50° C. for two hours and then stopped by heating to 70° C. for 1 hour or 100° C. for 5 minutes.

After cooling, the polymers with a high molecular weight are precipitated by adding HCl 1N and then eliminated by centrifugation, for example at 5000 g during 30 minutes, or by filtration.

Lastly, the pH of the supernatant is readjusted to a value comprised between 6 and 8, for example around 6.9.

The oligogalacturonides in compositions according to the invention preferably have a degree of polymerisation comprised between 1 and 5.

When the degree of polymerisation is equal to 1, oligogalacturonide refers to the galacturonic acid monomer entity that results from pectin hydrolysis.

When the degree of polymerisation is between 2 and 20, oligogalacturonide refers to an oligomer that consists of residues of galacturonic acid linked by 1-4 links. The number of galacturonic acid residues is 2 to 20, and preferably equal to 2, 3, 4 or 5.

The oligogalacturonides employed in the compositions according to the invention are not highly methylated or esterified. The compositions according to the invention comprise, in dry equivalent in relation to the overall weight of the composition, between 0.001% and 1% and preferably 0.01% of oligogalacturonides.

The cosmetic, food or dermatological compositions that employ the oligogalacturonides as their active agent can also contain other active substances, more particularly, plant extracts.

The compositions according to the invention can also contain one or several formulation agents or additives of common or conventional use in cosmetic, food and dermatological compositions such as, as non-exhaustive examples, demulcents, colorants, film-forming agents, surface active agents, perfumes, preservatives, emulsifiers, oils, glycols, sebum-absorbing agents, vitamins, etc. Thanks to this knowledge in the field of cosmetics, foodstuffs and dermatological products, those skilled in the art know which formulation agents to add to the compositions and what amounts in relation to the desired properties.

The compositions according to the invention can be presented in any form known to those skilled in the art of cosmetology and dermatology without any pharmaceutical restrictions other than application to the skin of the face or body. The compositions according to the invention that contain the oligogalacturonides as their active agent are advantageously presented mainly in the form of a gel, a cream, an emulsion, a milk or a spray for topical application. The compositions according to the invention can also be formulated for oral administration, mainly in the form of gelatin capsules, capsules, tablets, etc. Oral administration is preferred when the oligogalacturonides of the invention are used for foodstuffs. Thus, the present invention also relates to use of the oligogalacturonides as defined above, characterised in that the said oligogalacturonides are intended for oral administration.

The compositions of the invention can also be presented in the form of a patch, in which the oligogalacturonides are dispersed.

Other advantages and characteristics of the invention will emerge from the following examples concerning the preparation of oligogalacturonides and their use as a cosmetic agent.

### EXAMPLES

I—Preparation of oligogalacturonides.
   1) Operating Process.
Pectin: placed in a solution of 0.1% to 10% (1% of pectin HB AU910).
The pH is adjusted to 4.5, preferably using a solution of acetic acid.
Enzyme: a solution is prepared that corresponds to 10 to 1000 and preferably 100 units of polygalacturonase.
The enzymatic solution is added to the pectin solution in order to obtain at the end from 1 to 10 and preferably 4 units of pectinase.
Hydrolysis is performed at 50° C. for 2 hours, and then stopped by heating to 70° C. for 1 hour or 100° C. for 5 minutes.
After cooling, the polymers with a high molecular weight are precipitated by adding HCl 1N and then eliminated by centrifugation, for example at 5000 g during 30 minutes, or by filtration.
The pH of the supernatant is readjusted at the end to a value comprised between 6 and 8, for example around 6.9.
The oligogalacturonides formed are advantageously atomised or lyophilised at the end in order to enable better preservation and easier handling.
   2) HPLC Analysis of the Oligogalacturonides Formed.
An analysis technique producing a chromatographic profile of the oligogalacturonides formed was perfected.
Column: TSK gel DEAE 5-PW (TOSOHAAS)
Eluent: CH3COONH4 1M/H2O in elution gradient.
Mobile phase flow rate: 1 ml/min
Light diffusion evaporative detector (DEDL; ALTECH)
Oven temperature: 130° C.
Nitrogen flow rate: 4.0 SLPM (standard litre per minute): standard condition for H2O as a solvent.

The selection of gradient is presented in table 1 below:

TABLE 1

| Time in minutes | % A (CH3COONH4 1M) | % b (H2O) |
|---|---|---|
| 0 | 10 | 90 |
| 1 | 30 | 70 |
| 3 | 30 | 70 |
| 30 | 50 | 50 |
| 45 | 50 | 50 |

A distribution of the oligomers by size (dp: degree of polymerisation) from 1 to 5 is observed.
The lack of a standard does not allow quantitative determination of the oligomers of dp 4 and 5, but quantitative determination of the horter oligomers is possible.
If we proceed according to the conditions described of hydrolysis of a pectin solution with a concentration of 10 g/l, the total concentration of mono-, di- and trigalacturonic acid is around 4.5 g/l at the end, in other words, around 45% in terms of weight of the pectin placed in the solution.
II—Effects of oligogalacturonides on the differentiation of epidermal cells.
The following experiments were conducted on a reconstructed human skin model. This model consists of an equivalent dermis made up of a lattice of collagen contracted by normal human dermal fibroblasts (Bell E, Ivarsson B, Merril C; Proc. Natl. Acad. USA 1979, vol 76, no. 3, pp 1274-1278), which is epidermised in a second stage. The epidermisation is carried out by direct apposition of a biopsy of human skin (from plastic surgery) with a small diameter on the reconstituted dermal tissue (Coulomb B, Saiag P, Bell E, Breitburd F, Lebreton C, Heslan M, Dubertret L. Brit. Jour. Of Dermatol. 1986, 114, pp 91-101). Following the horizontal expansion of the epidermal cells, the assembly is placed in the air-liquid interface so as to induce the vertical phenomenon of differentiation. This reaches it optimum point after 7 to 10 days of culture in emersion.

This model has the advantage of comprising the two main types of skin cells, dermal cells and epidermal cells, and a part of the direct and indirect intercellular communications between these two types of cells. This model makes it possible to obtain a stratification of epithelial cells, which is almost the same as that of normal skin. Many structures (desmosomes, keratoyalin granules, stratum corneum, etc.) and proteins (different keratins, filaggrin, transglutaminases, etc.) bearing witness to this fact have been visualised using the suitable techniques. However, no structural elements that make up the dermal-epidermal basement membrane, such as the dense and clear layers that can be seen in electronic microscopy, or the associated proteins (laminins, collagen IV) have been detected between the dermal and epidermal compartments. Thus, this model does not include a dermal-epidermal junction and enables full communication between living cells (fibroblasts and keratinocytes).

The various efficiency tests carried out in the presence of a mix of oligogalacturonides have made it possible to prove the dose-dependent influence of this active agent on the keratinocyte differentiation process.

The use of a reconstructed human skin model according to Bell has made it possible to assess the gradual epithelialisation and the differentiation of the epidermal cells under the systematic influence of the mix of oligogalacturonides or in the absence of such mix. Two effects are detected according to the percentage of the active agent.

At concentrations of 0.001% to 1%, and particularly at 0.01%, the mix of oligogalacturonides reduces, in the first stage of epidermal reconstruction, the vertical expansion of the equivalent tissue compared to the untreated control. After 7 days of culture in emersion in the air-liquid interface, the equivalent epidermis has normal stratification. All the layers that are typical of a normal human epidermis are visible after histological colouring of the tissue (including the stratum corneum). The quality of stratification appears greatly increased in relation to that of the untreated control. In the view of the immunohistological approach, the basement layer has a normal cell-division rate (Ki67 approach), an improved distribution of the keratin 10 (keratin of the differentiated keratinocytes) which extends in the supra-basement layers; the involucrin markers are visible earlier in the epidermis. Only the stratum corneum seems to be less thick than in normal conditions.

This indicates that the presence of the oligogalacturonides mix at a concentration comprised between 0.001% and 1%, particularly 0.01%, makes it possible to restrict the vertical expansion of the epidermis in the benefit of improved stratification, and thus, improved cohesion inside the epidermis.

In this model in normal conditions, the natural desquamation is very low, due to the absence of friction or physical stress; it only takes place by means of enzymatic processes. It therefore emerges clearly that the phenomenon is improved by adding the mix of oligogalacturonides according to the invention. The stratum corneum of the epidermises reconstructed in the presence of the mix is therefore thinner. The action of the mix of oligogalacturonides on the desquamation is confirmed by the DNA micro-array technique using reconstructed epidermises marketed under the SkinEthic™ brand name. The mix of oligogalacturonides at 0.01% results in an increase of 214% of the DNA that codifies the stratum corneum chymotryptic enzyme (SCCE), which is responsible for the final degradation of the structural proteins of the stratum corneum and which triggers the desquamation.

Generally speaking, the presence of the mix of oligogalacturonides considerably improves the differentiation of keratinocytes as compared to an untreated control. The addition of 0.001% to 1%, preferably 0.01%, of the mix of oligogalacturonides has a beneficial effect on the epithelial organisation: it guarantees good cellular activity in the basement layer, better stratification of the epidermis and induces desquamation (gentle exfoliating action).

1-8. (canceled)

9. A method for preventing or treating skin disorders comprising administering to a subject a composition comprising oligogalacturonides.

10. The method according to claim 9, further comprising stimulating a differentiation of epidermal cells with said composition.

11. The method according to claim 10, wherein said stimulating step comprises stimulating a differentiation of keratinocytes.

12. The method according to claim 9, wherein said administering step comprises administering a dermatological composition.

13. The method according to claim 9, wherein said administering step comprises administering a composition which comprises, in dry equivalent, oligogalacturonides in a quantity between around 0.001% and around 1% in weight in relation to a total weight of the composition.

14. The method according to claim 9, wherein said administering step comprises administering a composition which comprises, in dry equivalent, oligogalacturonides in a quantity of around 0.01% in weight in relation to a total weight of the composition.

15. The method according to claim 9, wherein said administering step comprises administering a composition which comprises oligogalacturonides having a degree of polymerization comprised between around 1 and around 5.

16. The method according to claim 9, wherein said administering step comprises administering a composition which comprises oligogalacturonides obtained by hydrolysis of a pectin, said hydrolysis being performed by an enzymatic cocktail containing pectinases having mainly polygalacturonase, methyl esterase and polygalacturonase lyase activities.

17. The method according to claim 9, further comprising obtaining said oligogalacturonides by the following steps:
hydrolyzing of a pectin solution at a concentration of around 0.1% to around 10%, with a pH of around 4.5 by adding to said pectin solution an enzyme solution comprising around 10 to around 1000 units of polygalacturonase so as to obtain between around 1 and around 10 units of pectinase in a final solution;
stopping the hydrolysis; and
separating polymers with a high molecular weight and recovering the oligogalacturonides.

18. The method according to claim 17, wherein said hydrolysis stopping step comprises heating to around 70° C. for around 1 hour.

19. The method according to claim 17, wherein the separating step comprises precipitating the polymers with a high molecular weight by adding HCl 1N and then eliminating by centrifugation.

20. The method according to claim 19, further comprising readjusting the pH of the recovered supernatant to a value between around 6 and around 8.

21. The method according to claim 9, wherein said administering step comprises administering a composition which further comprises plant extracts.

22. The method according to claim 9, wherein said administering step comprises administering a composition which further comprises at least one formulation agent or additive selected from the group comprising demulcents, colorants, film-forming agents, surface active agents, perfumes, preservatives, emulsifiers, oils, glycols, sebum-absorbing agents, and vitamins.

23. The method according to claim 9, wherein said administering step comprises administering said composition in a form selected from the group comprising a gel, a cream, an emulsion, a milk and a spray for topical application.

24. The method according to claim 9, wherein said administering step comprises administering said composition in patch form.

25. The method according to claim 9, wherein said administering step comprises administering said composition in a form selected from the group comprising gelatin capsules, capsules, and tablets.

26. The method according to claim 9, wherein said administering step comprises administering said composition orally.

27. The method according to claim 9, wherein said administering step comprises administering said composition to improve appearance and feel of the skin.

28. The method according to claim 27, wherein said administering step comprises administering said composition to fight against skin irregularities.

29. The method according to claim 27, wherein said administering step comprises administering said composition to improve a radiance of a complexion.

30. The method according to claim 27, wherein said administering step comprises administering said composition to smooth facial skin.

31. The method according to claim 27, wherein said administering step comprises administering said composition to smooth body skin.

32. The method according to claim 27, wherein said administering step comprises administering said composition to improve skin tissue quality.

33. The method according to claim 27, wherein said administering step comprises administering said composition to stimulate a cell renewal process.

34. The method according to claim 33, wherein said administering step comprises administering said composition to stimulate an epidermal renewal process.
